(19) United States
(12) Patent Application Publication
(10) Pub. No.: US 2025/0382191 A1
(43) Pub. Date: Dec. 18, 2025

(54) Co-production of ammonia and methanol from biomass/MSW and natural gas

(71) Applicant: Massachusetts Institute of Technology, Cambridge, MA (US)

(72) Inventors: Daniel R. Cohn, Bridgton, ME (US); Leslie Bromberg, Sharon, MA (US); Emre Gencer, Medford, MA (US); Mohammad Ostadi, Somerville, MA (US)

(21) Appl. No.: 19/235,036

(22) Filed: Jun. 11, 2025

Related U.S. Application Data
(60) Provisional application No. 63/659,661, filed on Jun. 13, 2024.

(51) Int. Cl. C01C 1/04; C01B 3/02; C01B 3/34; C07C 29/151

(52) U.S. Cl. CPC C01C 1/0488; C01B 3/025; C01B 3/34; C07C 29/1518

(57) ABSTRACT

A method and apparatus is described that co-produces methanol and ammonia from biomass and natural gas. The carbon needed for the methanol process is derived from biomass, through oxygen gasification. The oxygen is produced by an air separation unit. The nitrogen from the air separation unit is used in an ammonia synthesis loop, combined with hydrogen from a natural gas pyrolysis unit. Methanol and ammonia are produced in parallel and independently. The combination has synergies that improve the efficiency and decrease the cost of the combined system, increase the flexibility of operation and reduce the emissions of global-warming gases.

This application claims priority of U.S. Provisional Patent Application Ser. No. 63/659, 661, filed Jun. 13, 2024, the disclosure of which is incorporated herein by reference in its entirety.

### BACKGROUND

In order to minimize emissions of Green House Gases, it is important for industry to maximize the use of byproducts and combine/integrate processes for minimization of resource requirements. The processes to be combined should have synergism that results in improved performance over separate systems. One potential candidate for combination are the generation of ammonia and methanol. Both ammonia and methanol are produced globally in large quantities.

The combination of ammonia/methanol systems have been described in the past. One earlier US Patent describes a system where the ammonia and methanol reactors are in series. The system does not need an air separation unit, while avoiding CO₂ emissions to the atmosphere. Another earlier US Patent describes a process in which methanol and ammonia are produced in parallel and independently. An earlier US Patent Publication describes methanol and ammonia coproduction in parallel utilizing an Air Separation Unit (ASU) to generate O₂ and N₂ for the process. The process syngas is generated in a common natural gas reformer. A more recent US Patent describes coproduction of methanol, ammonia and urea in parallel where an ASU is used to provide O₂ and N₂. Two different natural gas reformers are used to produce syngas in parallel, namely an autothermal reformer (ATR) and an steam methane reformer (SMR). Another US Patent describes coproduction of methanol and ammonia in parallel. An ATR is used to generate syngas for methanol synthesis while the syngas for the ammonia reactor is generated in water electrolyzer and an ASU. Part of the generated H₂ in electrolyzer is used to adjust the H₂ content of the syngas to methanol reactor. Another US Patent describes coproduction of methanol and ammonia in series. The reforming is performed with enriched-air, where the oxygen generated in water electrolyzer is mixed with air. Once-through methanol synthesis is carried out and the unconverted syngas is sent to ammonia reactor. Another US Patent describes coproduction of methanol and ammonia in parallel. A common reformer generated the required syngas for both processes. An ASU is used to produce the required O₂ and N₂. Another US patent describes coproduction of methanol and ammonia in series. A common reformer produces syngas for the process. A once-through methanol synthesis is performed, and the unconverted syngas is sent to ammonia reactor. The required N₂ is produced in an ASU. An older US Patent describes coproduction of methanol and ammonia in series. Syngas undergoes methanol production in two reactors which are placed in series. The syngas for the first methanol reactor is generated in a Steam reformer. The unconverted syngas in the first reactor is reformed in the second reformer utilizing air as the oxidizing agent. The unconverted syngas in the second methanol reactor is sent to Ammonia rector. Another US Patent describes coproduction of methanol and ammonia in parallel. Natural gas reforming is used to produce syngas for both processes. N₂ for the ammonia process is from reforming in air. Another US Patent describes coproduction of methanol and ammonia in series, while another older US Patent describes coproduction of methanol and ammonia in parallel. Two separate natural gas reformers, namely SMR and ATR produce syngas for methanol and ammonia production.

Finally, a US Patent describes coproduction of methanol and ammonia in parallel. Syngas is generated in two separate reformers while the N₂ required for ammonia synthesis is from an ASU.

However, an improved apparatus and method for the production of methanol and ammonia would be beneficial.

### SUMMARY

A method and apparatus is described that co-produces methanol and ammonia from biomass and natural gas. The carbon needed for the methanol process is derived from biomass, through oxygen gasification. The oxygen is produced by an air separation unit. The nitrogen from the air separation unit is used in an ammonia synthesis loop, combined with hydrogen from a natural gas or other hydrocarbon pyrolysis unit. Methanol and ammonia are produced in parallel and independently. The combination has synergies that improve the efficiency and decrease the cost of the combined system, increase the flexibility of operation and reduce the emissions of global-warming gases. This method and apparatus may have the following advantages:
Carbon-neutral production of ammonia and methanol from biomass/MSW and natural gas.
Cogeneration allows for synergies in process design and lower the financial risk of the plant through diversification of products
Economic benefits of co-production of ammonia and methanol:
   100% gain on an ASU
   30% gain on the pyrolyzer unit
   18% cost saving compared to two standalone units p2 No need for WGS reactors and CO₂ removal unit
Tops∅e estimates that investment cost compared to two standalone units is around 10-25% lower.
Production flexibility: Methanol prices often track global oil prices while ammonia and urea prices follow the agricultural market.
By addition of a few extra units (Ammonia reactor and compressors), Ammonia is co-produced.
Revenue of the process doubles while the investment increases by 74%

According to one embodiment, a process for the co-production of methanol and ammonia in parallel is disclosed. The method comprises using a hydrocarbon feedstock for pyrolysis to create a separate hydrogen stream; preparing a separate oxygen stream and a separate nitrogen stream by separation of air; introducing the separate oxygen stream into a gasifier; gasifying, in the gasifier, a biomass/MSW feedstock to produce synthesis gas (referred to as methanol syngas) comprising hydrogen and carbon oxides for methanol synthesis reaction; converting the methanol syngas to methanol in a methanol reactor; and in parallel introducing part of the separate hydrogen stream and the separate nitrogen stream into an ammonia synthesis reactor, and converting the nitrogen stream and hydrogen stream to ammonia.

According to another embodiment, a process for the co-production of methanol and ammonia in parallel is disclosed. The method comprises preparing a separate oxygen stream and a separate nitrogen stream by separation of air; preparing a separate hydrogen stream by use of a water/steam electrolyzer; introducing the separate oxygen stream into a gasifier; gasifying, in the gasifier, a biomass/MSW feedstock to produce synthesis gas (referred to as methanol syngas) comprising hydrogen and carbon oxides for methanol synthesis reaction; converting the methanol syngas to methanol in a methanol synthesis reactor; and in parallel introducing part of the separate hydrogen stream and the separate nitrogen stream into an ammonia synthesis reactor, and converting the nitrogen stream and hydrogen stream to ammonia.

According to another embodiment, a system for production of methanol and ammonia is disclosed. The system comprises an air separation unit to produce a separate oxygen stream and a separate nitrogen stream; a gasifier to receive a portion of the separate oxygen stream and biomass or municipal solid waste and generate methanol syngas; a pyrolyzer or an electrolyzer to produce a separate hydrogen stream; a methanol reactor to produce methanol from the methanol syngas; and an ammonia reactor to produce ammonia from a portion of the separate hydrogen stream and a portion of the nitrogen stream.

According to another embodiment, a process for the co-production of a Fischer Tropsch liquid fuel and ammonia in parallel is disclosed. The method comprises preparing a separate oxygen stream and a separate nitrogen stream by separation of air; preparing a separate hydrogen stream by use of a water/steam electrolyzer or a pyrolyzer; introducing the separate oxygen stream into a gasifier; gasifying, in the gasifier, a biomass/MSW feedstock to produce synthesis gas (referred to as syngas) comprising hydrogen and carbon oxides; converting the syngas to a Fischer Tropsch liquid fuel in a Fischer Tropsch reactor; and in parallel introducing part of the separate hydrogen stream and the separate nitrogen stream into an ammonia synthesis reactor, and converting the nitrogen stream and hydrogen stream to ammonia.

### BRIEF DESCRIPTION OF THE DRAWINGS

For a better understanding of the present disclosure, reference is made to the accompanying drawings, in which like elements are referenced with like numerals, and in which:

FIG. 1 shows parallel production of methanol and ammonia.

FIG. 2 shows parallel production of methanol and ammonia with intermediate storage of oxygen and nitrogen.

FIG. 3 shows parallel production of methanol and ammonia with intermediate storage of oxygen and nitrogen.

FIG. 4 shows parallel production of methanol and ammonia with process heat storage and intermediate storage of oxygen and nitrogen.

FIG. 5 shows parallel production of methanol and ammonia using a water electrolyzer.

### DETAILED DESCRIPTION

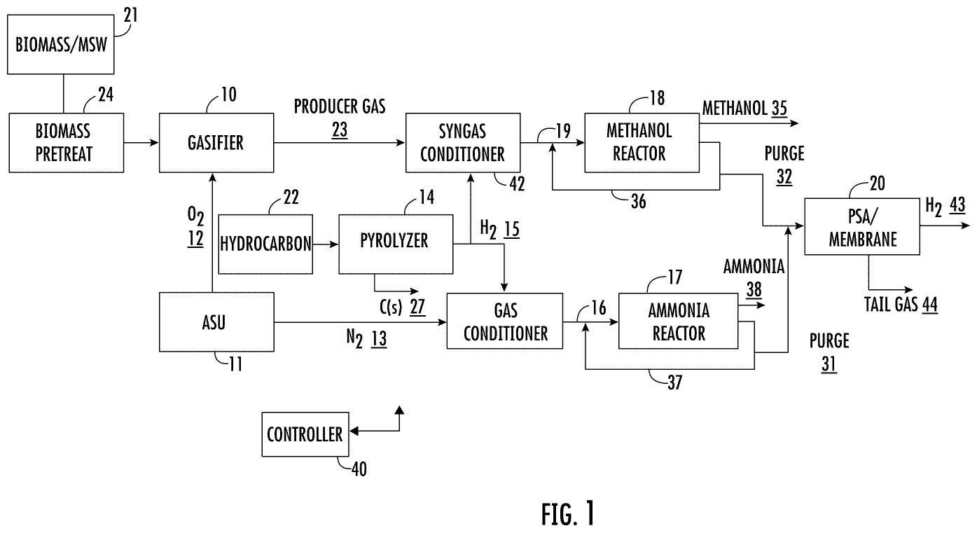

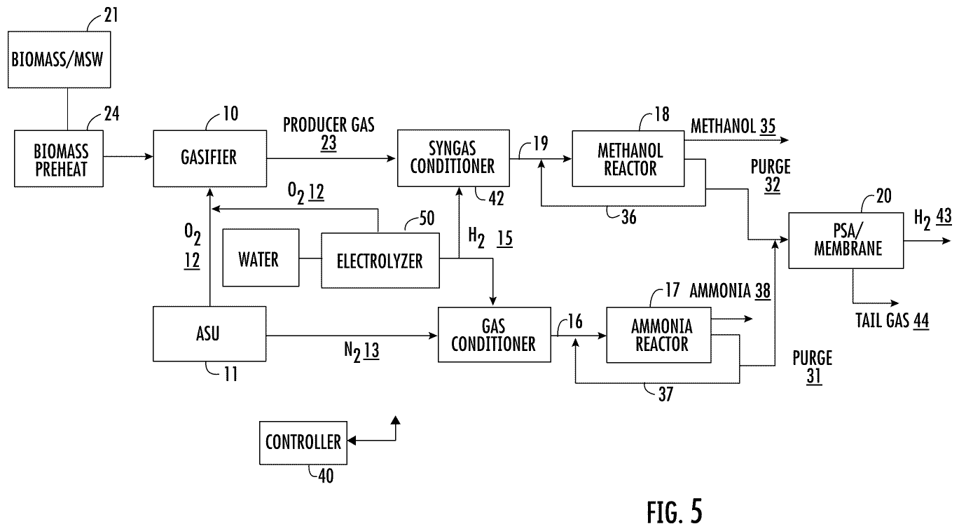

The present disclosure describes co-production of methanol and ammonia from pyrolysis of a hydrocarbon feedstock and biomass with low carbon emission. A first embodiment is shown in FIG. 1. The methanol syngas 19 for the methanol reactor 18 is produced from biomass gasification, using gasifier 10, with additional hydrogen from pyrolysis of a hydrocarbon feedstock 22 or a water/steam electrolyzer 50 (see FIG. 5) to adjust the stoichiometric ratio required for methanol synthesis. Methanol syngas 19 is a mixture of hydrogen and carbon oxides. Methanol 35 is industrially produced via catalytic conversion of methanol syngas 19 at pressures up to 250 bar and temperatures from ˜250° C. to 300° C. The methanol synthesis reaction is exothermic; the reactor temperature is often controlled by circulating boiling water as a coolant at elevated temperatures, such as 250° C. Due to low conversion per pass in the methanol reactor 18, a recycle stream or recycle loop 36 is required to increase the overall conversion of the methanol syngas 19. A pyrolyzer 14 and/or a water/steam electrolyzer 50 (see FIG. 5) are used to generate a hydrogen stream 15 with zero carbon emissions. The pyrolyzer 14 uses a hydrocarbon feedstock 22, such as natural gas. The separated carbon 27 from the pyrolyzer 14 is in solid form and is a valuable byproduct. The gasifier 10 utilizes oxygen 12 generated in an Air

Separation Unit (ASU) 11. The ASU 11 also generates a nitrogen stream 13. Part of the hydrogen stream 15 from the pyrolyzer 14 is mixed with the nitrogen stream 13 from the ASU 11 to generate ammonia syngas 16 for the ammonia synthesis reactor 17. An ammonia synthesis reactor 17 is a high-pressure chemical reactor designed to facilitate the catalytic reaction of nitrogen (N₂) and hydrogen (H₂) to form ammonia (NH₃) 38 according to an exothermic reaction. The reaction is typically carried out in the presence of an iron-based catalyst promoted with potassium, aluminum and calcium oxides. In some advanced designs, ruthenium-based catalysts are used for improved activity. The ammonia synthesis reactor 17 operates at high pressures (typically 100-250 bar) and elevated temperatures (300-500° C.) to balance reaction kinetics and equilibrium yield. Because the N₂ conversion is limited by equilibrium, a recycle stream 37 of unconverted ammonia syngas 16 is required to have an economically feasible process. The methanol reactor 18 and ammonia synthesis reactor 17 operate in parallel and independently, therefore increasing the flexibility of the process.

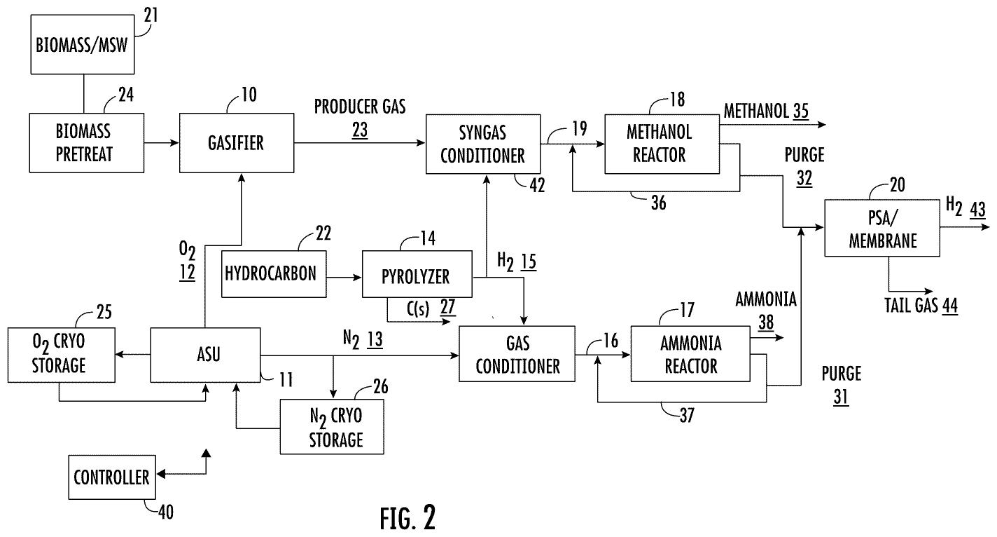

It may be possible for the plant to have both a pyrolyzer 14 and a water/steam electrolyzer 50 (see FIG. 5), with the option of selecting the combination that results in the lowest cost (depending on the cost of electricity and natural gas). If a water/steam electrolyzer 50 is used, the oxygen co-produced by the water/steam electrolyzer 50 may be used in the gasifier 10, as shown in FIG. 5, decreasing the demand of oxygen from the ASU 11. Excess nitrogen or oxygen can be stored cryogenically, as shown in FIGS. 2-4.

The present disclosure describes the parallel co-production of methanol 35 and ammonia 38 from biomass/MSW (municipal solid waste) 21. Biomass/MSW 21 has limited hydrogen content, not enough for the synthesis of methanol 35 or ammonia 38. The present system and method utilizes pyrolysis of natural gas or another hydrocarbon feedstock 22 for the generation of low carbon-intensity hydrogen (turquoise hydrogen), coupled to an air separation unit 11 for the generation of a nitrogen stream 13 and oxygen 12. The oxygen 12 from the air separation unit 11 is used for the preparation of methanol syngas 19 by oxygen gasification of a biomass/MSW feedstock 21 and the nitrogen stream 13 from the air separation unit 11 and the hydrogen stream 15 from the pyrolyzer 14 are used in a parallel process stage in synthesis of ammonia 38. A controller 40 may be used to coordinate the operation of all of the components described herein. The controller 40 may include a general purpose processing unit, a personal computer, a micro-controller or any suitable processing unit. That processing unit may be in communication with a memory device that contains instructions, which when executed by the processing unit, enable the controller to operate the system as described herein.

Conventionally, methanol is produced on an industrial scale from methanol synthesis gas, which is a mixture of varying amounts of H₂, CO, and CO₂ often derived from gasified coal or reformed natural gas.

Ammonia synthesis gas is conventionally prepared by mixing Nitrogen from an ASU and Hydrogen from steam reforming of natural gas or gasification of coal.

Recently, use of electrolysis of water for production of hydrogen and air separation to produce nitrogen has been proposed for the preparation of ammonia synthesis gas. The downside of this combination is that oxygen is a product of both ASU and water electrolysis, therefore it is produced in excess, which is considered as energy loss.

The present system and method uses the synergisms of biomass/MSW gasification using oxygen 12 from an air separation unit (ASU) 11, and creating ammonia 38 using the nitrogen stream 13 separated from the air stream. The hydrogen stream 15 from the pyrolyzer 14 (“turquoise hydrogen”) is used to supplement the synthesis gases for both methanol and ammonia synthesis. The nitrogen stream 13 from the ASU 11 is used in a parallel process for the preparation of ammonia syngas 16. In some embodiments, the hydrogen stream 15 and the nitrogen stream 13 are introduced into the ammonia synthesis reactor 17 in amounts to provide a molar ratio of the hydrogen 15 to the nitrogen 13 of 2.7-3.3. In some embodiments, the ASU 11 is powered using renewable energy, such as solar power.

Biomass or MSW 21 is the preferred feedstock to the process, as it contains high levels of biogenic carbon and thus has a small carbon footprint (and even negative footprint if the carbon is used in chemicals that do not result in CO₂ emissions). The biomass is gasified using an oxygen-partial oxidation process; however, it is possible to combine oxygen and steam in the gasifier 10 for an autothermal process.

The producer gas 23 produced by the gasifier 10 has hydrogen shortage for the production of methanol 35. The hydrogen may be produced from sources, several including pyrolysis of a hydrocarbon feedstock 22 or electrolysis. Natural gas pyrolysis is attractive because of the lower energy requirement compared to electrolysis. The carbon produced may be used in other processes or captured and stored; sequestration of solid carbon 27 is easier than the sequestration of CO₂. In some embodiments, the module (M=(H₂−CO)/(CO+CO₂)) of the methanol syngas 19 is adjusted to a value of between 1.9 and 2.2 using the syngas conditioner 42. Further, the input to the pyrolyzer 14 may be a hydrocarbon feedstock 22, including natural gas, methane and others.

The pyrolyzer 14 may be one of a molten metal or salt unit, a thermal unit (solar), chemical (i.e. combustion) or electrical (including plasmas). The pyrolyzer 14 may be powered by renewable energy or by burning part of the hydrogen stream 15 or by burning part of hydrocarbon feedstock 22.

The heat from the products of the pyrolyzer 14 (both the solid carbon 27 and the hydrogen gas 15) may be used for providing heat for the hydrocarbon feedstock 22 entering the pyrolyzer 14 or used elsewhere in the system, such as in the biomass pretreatment unit 24, in the gasifier 10, or/and in the distillation column for the raw methanol purification. The heat exchange can happen with either a regenerator or a recuperator. In particular, in some embodiments, the regenerator may use the hot carbon solids 27 from the pyrolysis (after separation from the gaseous products that include hydrogen) to preheat the hydrocarbon feedstock 22, which may be natural gas. Coking on the carbon solids 27 would assist with the pyrolyzer 14. Alternatively, or in addition, in the case of molten fluids in the pyrolysis process, the producer gas 23 from the gasifier 10 (which can be on the order of 1600° C., depending on the gasifier design) may be used to preheat the hydrocarbon feedstock 22 used in the pyrolyzer 14, decreasing the required external heat for the process. The hot or cooled solid carbon 27 from the pyrolyzer 14 may be used to treat the producer gas 23 from the gasifier 10 to help control tars from the producer gas 23. The products from the pyrolyzer 14 are much hotter than needed for the chemical synthesis, and the excess heat may be used elsewhere in the system, including preheating the biomass, natural gas and steam generation, or in the distillation column. Introducing a small amount of carbon solids 27 from the pyrolyzer 14 into the gasifier 10 may also improve the performance of the gasifier 10, at the cost of a small increase of the carbon footprint of the methanol 35 and ammonia 38. This approach may also be used during transients, such as startup or variation in throughput.

The exothermicity of the chemical synthesis processes may be used elsewhere in the system. The excess heat can be used to preheat the biomass 21 and/or the hydrocarbon feedstock 22 used in the pyrolyzer 14 and/or steam generation, in the distillation column and generating steam.

It is also possible to use excess CO₂ from the gasifier 10 to produce urea from the ammonia. Since the CO₂ from the gasifier 10 is biogenic, its release at the end use of the urea has no Greenhouse Gas (GHG) impact. In the case of MSW, only a fraction of the CO₂ produced is biogenic and the release of the CO₂ at the end of use will have a finite GHG impact.

In addition, having a plant that produces both ammonia and methanol can be used in the synthesis of methylamines.

The biomass/MSW 21 may be torrefied before the gasification using a biomass pretreatment unit 24. Torrefaction improves the grindability of the material and can result in improved gasification. Biomass torrefaction and gasification may be integrated and is preferred, resulting in less loss of carbon from the process.

The excess heat from the pyrolyzer 14 or gasifier 10 may be used in the torrefaction reactor. The excess heat generated in water electrolysis (such as Proton Exchange Membrane and Alkaline Electrolysis) can be used for heating other streams in the process. The temperature of hot streams can be elevated by use of a heat pump to improve the heat quality.

It is possible to have synergies between the two chemical synthesis loops. For example, the tail gas from the ammonia synthesis reactor 17 may be used to increase the hydrogen needed for the methanol 35. The high-pressure ammonia tail gas (purge) 31 from the ammonia synthesis reactor 17, mostly hydrogen and nitrogen, with small amounts of ammonia, may be used in the gasifier 10, or downstream of the gasifier 10 in the methanol recycle loop 36. However, it is not possible to use the tail gas (purge) 32 from the methanol reactor 18 in the ammonia synthesis, without having separated all the oxygen containing compounds. The methanol tail gas 32 from the methanol reactor can instead be used in the gasifier 10.

It is possible to use a membrane or pressure swing adsorption (PSA) 20 to separate the hydrogen 43 from the ammonia tail gas 31 and/or from the methanol tail gas 32. The hydrogen 43 may be used in either of the recycle loops. The tail gas 44 may be combusted and part of the heat may be used in other parts of the system. The membrane can be part of a membrane reactor, with a system that includes a water gas shift catalyst to convert unreacted CO into hydrogen. The hydrogen is being continuously removed from the system by the membrane, increasing the hydrogen 43 production. Steam may have to be added to the membrane or pressure swing adsorption (PSA) 20.

In some embodiments, as shown in FIGS. 2-4, cryogenic separation of air may be preferred, as it produces simultaneously high purity oxygen and nitrogen. Because cryogenic separation is used, it is possible to cryogenically store the excess gasses, such as cryogenic storage of oxygen 25, cryogenic storage of nitrogen 26 or even cryogenic storage of hydrogen, to be used under conditions where the air separation unit 11 is not available, or the cost of electricity is high. Hydrogen may be stored at temperatures above the boiling point of the hydrogen, at high pressure, decreasing the pressure requirement for room temperature storage. Note that, while not shown, the water/steam electrolyzer 50 of FIG. 5 may be used in any of these embodiments as well. The controller 40 may be used to determine whether the water/steam electrolyzer 50 or the pyrolyzer 14 are used, depending on the cost of electricity, the cost of hydrocarbon feedstock 22, as well as forecasted costs (that is using costs at the time as well as forward costs, as there is a cost associated with shifting from one of the units to the other).

Renewable electricity is preferred, in order to decrease the carbon intensity of the products. Means of address the intermittency of the electricity is described below, using storage of reagents or products, in potential combination with batteries or generators. In addition, a fraction of the reagents or products may be stored for use to complement the power requirements using power conversion equipment on-site.

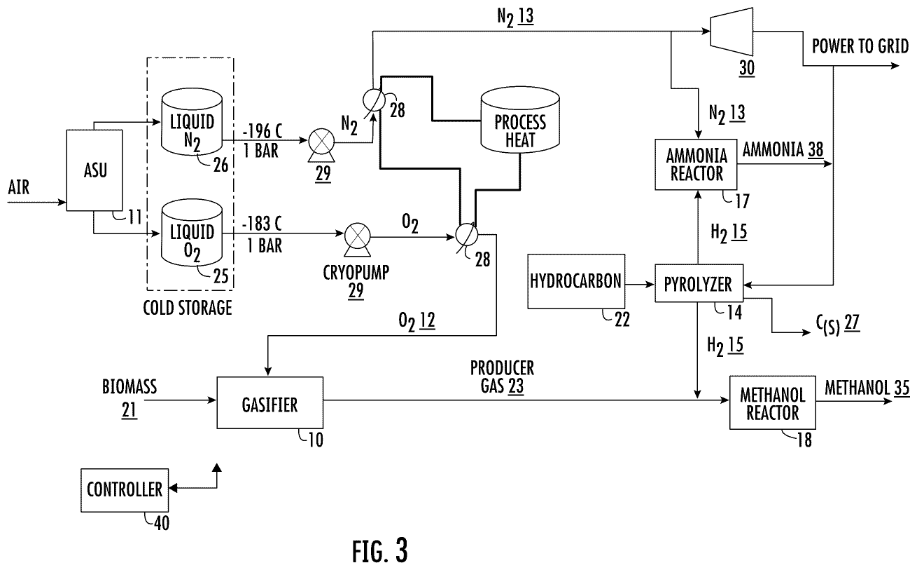

During times when the stored reagents are warmed up, excess heat and ambient air can be used to bring the temperature to those needed for the process, with pressurization when in the liquid state. For example, as shown in FIG. 3, process heat may be used to warm the nitrogen 26 and oxygen 25. This may be done using regenerators 28. Additionally, cryopumps 29 may be used to deliver the required oxygen 25 and nitrogen 26 to the system. The energy required for pressurization is decreased if the pressurizations are done when the feedstocks are liquid at cryogenic temperatures.

It is possible to supplement the power required for the process during those times when the cryogens are being retrieved, by running a Rankine cycle, especially since the gases need to be at high temperature. However, there is a decrease in pressure in the turbine expander 30 used to produce electricity. The turbine expander 30 should be used only in the case when the requirement for the chemical synthesis is lower than the capacity for pressurizing the liquid cryogens. The mass flow rate of oxygen is, of course, lower than the mass flow rate of nitrogen.

During the cryogen retrieval period, the cryogens may be pressurized at or at higher pressure than required by the chemical synthesis process. By pressurizing cryogens when liquid, the power requirement for compression is substantially decreased.

The performance of the system may be optimized by the use of thermal storage. During the retrieval of the liquid cryogens, a regenerator 28 may be used to warm the flow. The mass of the regenerator 28 is cooled as the cryogens warm up. The regenerator 28 may be a device with substantial heat transfer, in order to minimize temperature gradients and minimize entropy generation. It can either vary temperature or alternatively, use a phase-change material in order to allow isothermal energy transfer. Multiple regenerators can be used along the cryogenic loop, operating at different temperatures. During the reverse time, when fresh air is being chilled, the regenerators 28 may be used to help cool the gas and minimize the power requirement. The regenerators 28 need to be well insulated from the ambient environment in order to minimize thermal transfer and maximize the efficiency of the system. It is best if the thermal conduction along the direction of the gas flow is small, resulting in high thermal gradients along this direction. However, it is best if the thermal gradient in the perpendicular direction is small. Although not shown, FIG. 3 may include the biomass pretreatment unit 24, the conditioner for methanol syngas 19, the recycle loops, the gas conditioner and the pressure swing adsorption (PSA) 20, as described in FIG. 1. Alternatively or additionally, a water/steam electrolyzer 50 may be used.

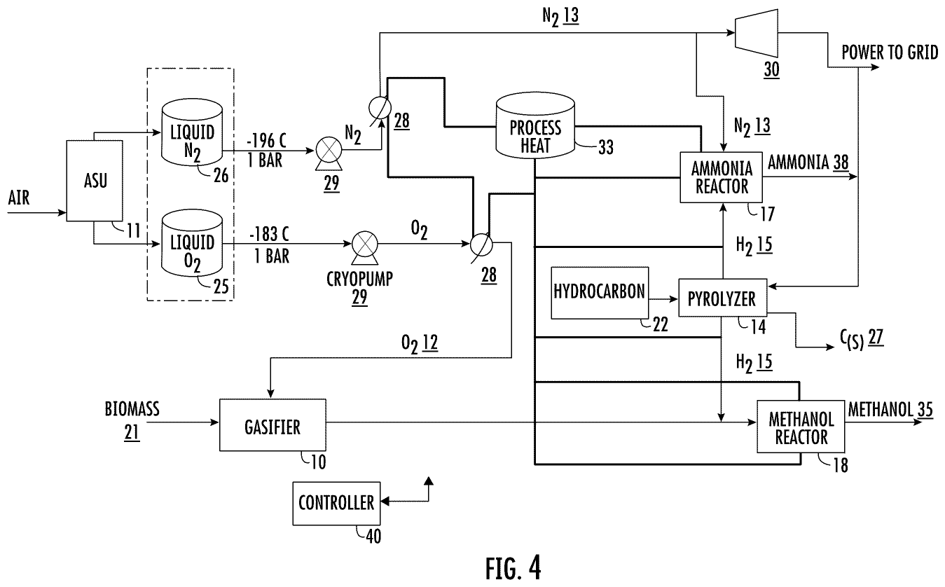

The system may also use thermal process energy storage, as shown in FIG. 4. The heat from the gasifier 10, the pyrolyzer 14 and/or the synthesis reactors 17, 18 may be stored in a thermal storage unit 33. As noted above, the excess heat generated in water electrolysis (such as Proton Exchange Membrane and Alkaline Electrolysis) can be used for heating other streams in the process. Similarly, the high temperature from the producer gas 23 from the gasifier 10, the hydrogen 15 and carbon 27 from the pyrolyzer 14 may also be used elsewhere in the process. The thermal energy may be used during conditions when some of the feedstocks are not available, for example, in the pyrolyzer 14, in the distillation unit, in the biomass or pyrolysis unit preheating, or during the recovery of the cryogenic oxygen or nitrogen.

The startup of the plant includes the following main steps:
1—Filling the O₂ and N₂ cryo storage tanks to a minimum level.
2—Running the torrefaction and the gasifier 10 to reach steady state and desired temperature.
3—Heating up the pyrolysis molten media through electrical heating or heat exchange with the gasifier 10, and injecting the first doses of hydrocarbon feedstock to convert to hydrogen.
4—Injecting the first doses of syngas to the methanol and ammonia synthesis reactors.

Another advantage of parallel ammonia and methanol synthesis is in the case of feedstock fluctuations and transients. As the methanol reactor 18 and ammonia synthesis reactor 17 are completely independent, each can be turned up and down independently.

Having cryogenic storage of O₂ and N₂ allows smoother operation. Moreover, the economy of the process is improved by storing the reagents when electricity is inexpensive.

Ammonia synthesis reactors 17 and methanol reactors 18 may be more flexible by having a staged reactor concept. Instead of having a big reactor, it may be divided into 2 or 3 stages. Bypassing one or more reactor stages allows the ability to adjust the conversion of reactors in case of transient inlet flow to reactors. In case of methanol, intermediate cooling, and separation of products between stages prolonged the catalyst lifetime and increases reactor productivity.

The pyrolyzer 14 may be split into several smaller tanks instead of having a large pyrolyzer. This improves the handling of process transients as well as redundancy of the process. All tanks need to be kept at temperature in order to allow quick startup. This can be done through electrical heating or heat exchange with the gasifier 10, or burning part of hydrocarbon, hydrogen, or part of the syngas.

In order to quantify the benefits of co-production of methanol and ammonia, a comparison is made with a standalone biomass-to-methanol process, as shown in Table 1. In the coproduction case, the total equipment cost increased by 74% while the revenue more than doubled. The revenue is calculated assuming equal price of 500 $/tonne for both methanol and ammonia. The production of ammonia and methanol are adjusted in order to maximize profits, with synthesis of one of products (either methanol or ammonia) increased when the relative costs of that product is higher. The flexibility is enabled by having reactors that can tolerate transients.

TABLE 1
Comparison of standalone methanol and coproduction
of ammonia and methanol from biomass

| | | Biomass | Percent |
|---|---|---|---|
| | Biomass to | to Methanol | increase |
| | Methanol | & Ammonia | (%) |
| Biomass 5% wet (tonne/hr) | 39.2 | 39.2 | |
| Natural Gas (kmol/hr) | 1252 | 4252 | 240 |
| Methanol (tonne/hr) | 48.4 | 48.4 | |
| Ammonia (tonne/hr) | 0 | 62.1 | |
| Carbon black (tonne/hr) | 30.1 | 102.1 | 240 |
| H₂ required for methanol (kmol/h) | 2503 | 2503 | |
| H₂ required for ammonia (kmol/h) | — | 6000 | |
| Total H₂ required (kmol/h) | 2503 | 8503 | 240 |
| O₂ from ASU (kmol/h) | 631.4 | | |
| N₂ from ASU generated (kmol/h) | 2375.3 | | |
| N₂ Needed (kmol/h) | 2000 | | |
| Excess N₂ (kmol/h) | 375.3 | | |
| Purchased equip. cost (M$) | 206.6 | 358.5 | 74 |
| Revenue ($/hr) | 24200 | 55250 | 228 |

While the production of methanol is described, it is understood that this system has other uses as well. For example, the process is also applicable to the production of other liquid fuels using Fischer Tropsch systems, which also require H₂ to CO ratios of 2. In this embodiment, the methanol reactor 18 is replaced with a Fischer Tropsch reactor.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A process for co-production of methanol and ammonia in parallel, comprising:
using a hydrocarbon feedstock for pyrolysis to create a separate hydrogen stream;
preparing a separate oxygen stream and a separate nitrogen stream by separation of air;
introducing the separate oxygen stream into a gasifier;
gasifying, in the gasifier, a biomass/MSW feedstock to produce synthesis gas (referred to as methanol syngas) comprising hydrogen and carbon oxides for methanol synthesis reaction;
converting the methanol syngas to methanol in a methanol reactor; and in parallel introducing part of the separate hydrogen stream and the separate nitrogen stream into an ammonia synthesis reactor, and converting the nitrogen stream and hydrogen stream to ammonia.

2. The process of claim 1, wherein a module (M=(H₂−CO)/(CO+CO₂)) of the methanol syngas is adjusted to a value of between 1.9 and 2.2 by adding a part of the separate hydrogen stream into the methanol syngas.

3. The process of claim 1, wherein the separate hydrogen stream and the separate nitrogen stream are introduced into the ammonia synthesis reactor in amounts to provide a molar ratio of hydrogen to nitrogen of 2.7-3.3.

4. The process of claim 1, wherein the pyrolysis of the hydrocarbon feedstock is powered by renewable energy or by burning part of the separate hydrogen stream or by burning part of the hydrocarbon feedstock.

5. The process of claim 1, wherein the separation of air is powered by renewable energy.

6. The process of claim 1, wherein the air is separated cryogenically.

7. The process of claim 6, wherein excess oxygen and excess nitrogen are cryogenically stored.

8. The process of claim 7, wherein process heat is used to warm cryogenically stored gasses.

9. A process for co-production of methanol and ammonia in parallel, comprising:
preparing a separate oxygen stream and a separate nitrogen stream by separation of air;
preparing a separate hydrogen stream by use of a water/steam electrolyzer;
introducing the separate oxygen stream into a gasifier;
gasifying, in the gasifier, a biomass/MSW feedstock to produce synthesis gas (referred to as methanol syngas) comprising hydrogen and carbon oxides for methanol synthesis reaction;
converting the methanol syngas to methanol in a methanol synthesis reactor; and in parallel introducing part of the separate hydrogen stream and the separate nitrogen stream into an ammonia synthesis reactor, and converting the nitrogen stream and hydrogen stream to ammonia.

10. The process of claim 9, wherein a module (M=(H₂−CO)/(CO+CO₂)) of the methanol syngas is adjusted to a value of between 1.9 and 2.2 by adding a part of the separate hydrogen stream into the methanol syngas.

11. The process of claim 9, wherein the separate hydrogen stream and the separate nitrogen stream are introduced into the ammonia synthesis reactor in amounts to provide a molar ratio of hydrogen to nitrogen of 2.7-3.3.

12. The process of claim 9, wherein the separation of air is powered by renewable energy.

13. The process of claim 9, wherein the air is separated cryogenically.

14. The process of claim 13, wherein excess oxygen and excess nitrogen are cryogenically stored.

15. The process of claim 14, wherein process heat is used to warm cryogenically stored gasses.

16. The process of claim 9, wherein the separate hydrogen stream is also preparing by pyrolysis of a hydrocarbon feedstock.

17. A system for production of methanol and ammonia comprising:
an air separation unit to produce a separate oxygen stream and a separate nitrogen stream;
a gasifier to receive a portion of the separate oxygen stream and biomass or municipal solid waste and generate methanol syngas;
a pyrolyzer or an electrolyzer to produce a separate hydrogen stream;
a methanol reactor to produce methanol from the methanol syngas; and
an ammonia reactor to produce ammonia from a portion of the separate hydrogen stream and a portion of the nitrogen stream.

18. The system of claim 17, wherein the pyrolyzer is used to produce the separate hydrogen stream, and solid carbon is also produced.

19. The system of claim 17, wherein the electrolyzer is used to produce the separate hydrogen stream, and a second separate oxygen stream is also produced.
